(19) United States
(12) Patent Application Publication
(10) Pub. No.: US 2013/0023696 A1
(43) Pub. Date: Jan. 24, 2013

(54) PROCESS FOR PRODUCING SULFUR-CONTAINING AMINO ACIDS

(71) Applicant: Koji Hagiya, Ibaraki-shi (JP)

(72) Inventor: Koji Hagiya, Ibaraki-shi (JP)

(73) Assignee: SUMITOMO CHEMICAL COMPANY, LIMITED, Chuo-ku, Tokyo (JP)

(21) Appl. No.: 13/639,210

(22) PCT Filed: Apr. 5, 2011
(86) PCT No.: PCT/JP2011/058957

§ 371 Date: Oct. 4, 2012

(30) Foreign Application Priority Data
Apr. 6, 2010 (JP) 2010-087564

(51) Int. Cl. C07C 51/305

(52) U.S. Cl. 562/526

(57) ABSTRACT

The present invention relates to a process for producing a sulfur-containing amino acid, comprising a step of oxidizing a 2-aminoethanol compound having, at position 2, a sulfur-containing hydrocarbon group having 1 to 24 carbon atoms in the presence of copper and water.

### TECHNICAL FIELD

The present application is filed, claiming the priorities based on the Japanese Patent Application No. 2010-087564 (filed on Apr. 6, 2010), and a whole of the contents of the application is incorporated herein by reference.

The present invention relates to a process for producing a sulfur-containing amino acid.

### BACKGROUND ART

Sulfur-containing amino acids such as methionine and S-alkyl cysteine exist commonly in the all organisms, and they are useful components for many important biological reactions. Particularly, methionine is an essential amino acid, which is an important compound for use as a feed additive.

For example, the following method is disclosed in “industrial organic chemistry”, Tokyo Kagaku-Dojin, 1978, pp. 273-275: 3-(methylthio)propionaldehyde obtained by addition of methanethiol to acrolein is reacted with hydrogen cyanide to obtain 2-hydroxyl-4-methylthiobutyronitrile; and then, the 2-hydroxyl-4-methylthiobutyronitrile is reacted with ammonium carbonate to obtain a substituted hydantoin and thereafter, the substituted hydantoin is hydrolyzed with an alkali. In addition, the following method is disclosed in “Chem. Ber.”, vol. 121, 1988, pp. 2209-2223: methanethiol is added to methyl 2-chloroacrylate; and then, the resultant adduct is reacted with a sodium azide and thereafter, the resultant product is hydrogenated under acidic conditions.

### DISCLOSURE OF INVENTION

### Problem to be Solved by the Invention

However, the methods disclosed in the above documents require using hydrogen cyanide or sodium azide as a raw material. However, these compounds require careful handling.

Under such a circumstance, there has been demanded a new process for producing sulfur-containing amino acids without using of hydrogen cyanide or sodium azide.

### Means for Solving the Problem

As a result of the present inventors' intensive studies for solving the above-described problem, the present invention is accomplished.

The present invention provides the followings:
[1] A process for producing a sulfur-containing amino acid, comprising a step of oxidizing a 2-aminoethanol compound having, at position 2, a sulfur-containing hydrocarbon group having 1 to 24 carbon atoms in the presence of copper and water.
[2] The process according to the above item [1], wherein the above-described step of oxidizing the 2-aminoethanol compound is carried out further in the presence of at least one typical metal compound selected from the group consisting of alkali metal compounds and alkaline earth metal compounds.
[3] The process according to the above item [2], wherein the above-described typical metal compound is at least one compound selected from the group consisting of alkali metal hydroxides and alkaline earth metal hydroxides.
[4] The process according to any one of the above items [1] to [3], wherein the sulfur-containing hydrocarbon group has no non-aromatic multiple bond.
[5] The process according to any one of the above items [1] to [4], wherein the 2-aminoethanol compound is 2-amino-4-methylthio-1-butanol.

According to the present invention, a new process for producing the sulfur-containing amino acids without using as a raw material any hydrogen cyanide or sodium azide which requires careful handling can be provided.

### MODES FOR CARRYING OUT THE INVENTION

Hereinafter, the present invention will be described in detail.

The 2-aminoethanol compound has a sulfur-containing hydrocarbon group at position 2 (the 2-aminoethanol compound is sometimes referred to as “alcohol compound”), for example, which is represented by the following formula:

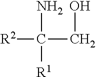

In the formula, R¹ and R² independently represent a sulfur-containing hydrocarbon group or a hydrogen atom, one of which represents the sulfur-containing hydrocarbon group. Herein, the sulfur-containing hydrocarbon group means a group comprising a sulfur atom, a carbon atom and a hydrogen atom. The hydrogen atom in the sulfur-containing hydrocarbon group may be substituted by a group inactive to an oxidation reaction as will be described later.

There is no limit in selection of the sulfur-containing hydrocarbon group if the group has 1 to 24 carbon atoms. The group may be a saturated sulfur-containing hydrocarbon group having no multiple bond, or a unsaturated sulfur-containing hydrocarbon group having a double bond and/or a triple bond. The unsaturated sulfur-containing hydrocarbon group may contain an aromatic isocyclic ring such as a benzene ring and/or an aromatic heterocyclic ring such as a thiophene ring.

The saturated sulfur-containing hydrocarbon group may be linear, branched or cyclic. Hereinafter, the linear or branched saturated sulfur-containing hydrocarbon group is sometimes referred to as a saturated chain sulfur-containing hydrocarbon group. The cyclic saturated sulfur-containing hydrocarbon group is sometimes referred to as a saturated cyclic sulfur-containing hydrocarbon group.

The saturated chain sulfur-containing hydrocarbon group includes a methylthiomethyl group, an ethylthiomethyl group, a propylthiomethyl group, an isopropylthiomethyl group, a tert-butylthiomethyl group, a 1-(methylthio)ethyl group, a 2-(methylthio)ethyl group, a 1-(ethylthio)ethyl group, a 2-(ethylthio)ethyl group, a 1-(propylthio)ethyl group, a 2-(propylthio)ethyl group, a 2-(isopropylthio)ethyl group, a 2-(tert-butylthio)ethyl group, a 1-(methylthio)propyl group, a 2-(methylthio)propyl group, a 3-(methylthio)propyl group, a 3-(ethylthio)propyl group, a 3-(propylthio)propyl group, a 3-(isopropylthio)propyl group and a 2,3-(dimethylthio)propyl group.

The saturated cyclic sulfur-containing hydrocarbon group includes a cyclopropylthiomethyl group, a cyclobutylthiomethyl group, a cyclopentylthiomethyl group, a cyclohexylthiomethyl group, a 2-(methylthio)cyclopropyl group, a 2-(methylthio)cyclobutyl group, a 2-(methylthio)cyclopentyl group, a 2-(methothio)cyclohexyl group, a 4-(methylthio)cyclohexyl group, a 2-methyl-4-(methylthio)cyclohexyl group, a 2,4-(dimethylthio)cyclohexyl group, a 2-thiacyclohexyl group and 4-thiacyclohexyl group.

The unsaturated sulfur-containing hydrocarbon group includes a vinylthiomethyl group, a 1-(vinylthio)ethyl group, a 2-(vinylthio)ethyl group, a 4-methylthio-1-butenyl group, a 4-methylthio-2-butenyl group, a 2-methylthiophenyl group, a 3-methylthiophenyl group, a 4-methylthiophenyl group, a 2-methyl-4-methylthiophenyl group, a 2,4-(dimethylthio)phenyl group, a phenylthiomethyl group, a 1-(phenylthio)ethyl group, a 2-(phenylthio)ethyl group, a benzylthiomethyl group, a 1-(benzylthio)ethyl group, a 2-(benzylthio)ethyl group, a 2-thienyl group, a 3-thienyl group and a 2-methyl-3-thienyl group.

The group inactive to an oxidation reaction includes C₁₋₁₂ alkyloxy groups such as a methoxy group, an ethoxy group, a propyloxy group, an isopropyloxy group, a butyloxy group, an isobutyloxy group, a sec-butyloxy group, a tert-butyloxy group, a pentyloxy group and a hexyloxy group;
C₇₋₁₂ aralkyloxy groups such as a benzyl group, etc.;
C₃₋₈ cycloalkyloxy groups such as a cyclopropyloxy group, a cyclobutyloxy group, a cyclopentyloxy group and a cyclohexyloxy group;
C₆₋₁₂ aryloxy groups such as a phenoxy group, a 2-methylphenoxy group, a 4-methylphenoxy group and a 4 phenylphenoxy group;
C₁₋₆ perfluoroalkyloxy groups such as a trifluoromethoxy group and a pentafluoroethoxy group;
substituted or unsubstituted amino groups, among which the substituted amino group has usually 1 to 12 carbon atoms, such as an amino group, a methylamino group, a dimethylamino group, a benzylamino group, a tert-butoxycarbonylamino group and a benzyloxycarbonylamino group;
C₂₋₁₂ acyl groups such as an acetyl group, a propionyl group, a butylyl group, an isobutylyl group, a valeryl group, an isovaleryl group, a pivaloyl group and a benzoyl group;
C₂₋₁₂ acyloxy groups such as an acetyloxy group, a propionyloxy group, a butylyloxy group, an isobutylyloxy group, a valeryloxy group, an isovaleryloxy group, a pivaloyloxy group and a benzoyloxy group; and
halogen atoms such as a fluorine atom and a chlorine atom.

The hydrogen groups of C₆₋₁₂ aryloxy groups and C₇₋₁₂ aralkyloxy groups may be substituted by at least one selected from the group consisting of C₁₋₁₂ alkyloxy groups, C₆₋₁₂ aryloxy groups, halogen atoms and the like.

The sulfur-containing hydrocarbon group is preferably a saturated sulfur-containing hydrocarbon group having no multiple bond, more preferably a saturated chain sulfur-containing hydrocarbon group, still more preferably a 2-(C₁₋₄₂ alkylthio)(C₁₋₆ alkyl) group, particularly preferably a 2-(methylthio)ethyl group.

The alcohol compound includes specifically 2-amino-3-methylthio-1-propanol, 2-amino-3-tert-butylthio-1-propanol, 2-amino-3-benzylthio-1-propanol, 2-amino-3-ethylthio-1-propanol, 2-amino-4-methylthio-1-butanol, 2-amino-4-ethylthio-1-butanol, 2-amino-4-propylthio-1-btanol, 2-amino-4-benzylthio-1-butanol, 2-amino-5-methylthio-1-pentanol, 2-amino-5-ethylthio-1-pentanol, 2-amino-5-propylthio-1-pentanol and 2-amino-5-benzylthio-1-pentanol, preferably 2-amino-4-methylthio-1-butanol.

As the alcohol, a commercially available product may be used, and also, the alcohol produced by using any known method such as a method by reacting an ethylene oxide having a sulfur-containing hydrocarbon group with ammonia (e.g., Izvestiya Akademii Nauk SSSR, Seriya Khimicheskaya, vol. 9, pp 2090-2094, 1985) or the like.

The alcohol compound is oxidized in the presence of copper (hereinafter the copper sometimes referred to as a copper catalyst) and water. Hereinafter, the reaction of oxidizing the alcohol compound in the presence of the copper catalyst and water is sometimes referred to as an oxidation reaction or the present reaction. The alcohol compound is converted to a sulfur-containing amino acid by the present reaction.

The copper catalyst may be one in which a copper metal is supported on a support (hereinafter sometimes referred to as a supported catalyst) or may be one in which a copper metal is not supported on a support. The copper catalyst may be a developing catalyst. The developing catalyst, in other words “sponge catalyst”, is a compound obtained by treating a copper-containing alloy with an acid or an alkali. By the treatment, the components other than copper are removed from the alloy to obtain, so that the developing catalyst is generally composed of copper which forms a sponge-like structure having numerous pores. The copper-containing alloy includes an alloy composed of copper and aluminum and an alloy composed of copper and silicon. The copper catalyst may be a catalyst obtained as follows: at least one copper salt selected from the group consisting of copper nitrates, copper sulfates, copper formates, copper acetates, copper carbonates, copper halides, copper hydroxides and copper oxides is reduced with a reducing agent such as hydrazine or hydrogen.

The support includes at least one selected from the group consisting of an activated carbon, alumina, silica, zeolite, diatomaceous earth and zirconium oxide. It is preferable that the support has a larger surface area in order to improve reactivity. The supported catalyst may be commercially available product, or may be a catalyst obtained as follows: copper metal or an alloy of copper with aluminum is supported on the above-described support to obtain the supported catalyst. Otherwise, the supported catalyst may be a catalyst obtained as follows: at least one salt selected from the group consisting of copper nitrates, copper sulfates, copper formates, copper acetates, copper carbonates, copper halides, copper hydroxides and copper oxides is supported on the above-described support by coprecipitation process or impregnation process, and then this supported salt is reduced with hydrogen or is calcined.

The copper catalyst is preferably a developing catalyst or a supported catalyst, more preferably a developing catalyst.

The amount of the copper catalyst to be used may vary depending on the form of the copper catalyst in use. The amount of the copper catalyst to be used is preferably 0.5 mole or less per mole of the alcohol compound from an economical viewpoint. When the copper catalyst is a supported catalyst, the amount of the catalyst including the support is usually from 0.1 to 100 parts by weight per part by the weight of the alcohol compound.

The amount of water to be used is preferably one mole or more per mole of the alcohol compound. The upper limit is not limited, but it is usually 200 moles per mole of the alcohol compound.

Preferably, the present reaction is carried out further in the presence of at least one typical metal compound selected from the group consisting of alkali metal compounds and alkaline earth metal compounds.

Examples of the alkali metal compounds include alkali metal carbonates such as sodium carbonate, sodium bicarbonate, potassium carbonate, potassium bicarbonate, lithium carbonate and lithium bicarbonate; and alkali metal hydroxides such as sodium hydroxide, potassium hydroxide and lithium hydroxide.

Examples of the alkaline earth metal compounds include alkaline earth metal carbonates such as magnesium carbonate and calcium carbonate; and alkaline earth metal hydroxides such as magnesium hydroxide and calcium hydroxide.

The typical metal compound is preferably at least one compound selected from the group consisting of alkali metal hydroxides and alkaline earth metal hydroxides, more preferably alkali metal hydroxides, still more preferably sodium hydroxide.

The amount of the typical metal compound to be used is preferably one mole or more per mole of the alcohol compound, while the upper limit thereof is not limited. The amount of the typical metal compound to be used is 2 moles or less per mole of the alcohol compound from a practical viewpoint.

The present reaction may be carried out further in the presence of an organic solvent.

There is no limit in selection of the organic solvent if it does not hinder the present reaction. Examples of the organic solvent include ester solvents such as ethyl acetate, and nitrile solvents such as acetonitrile and propionitrile.

The amount of the organic solvent to be used is, while not limited, practically 100 parts by weight or less per part by weight of the alcohol compound.

In the present reaction, the order of blending the reactants is not limited. For example, in a preferred mode, the alcohol compound, the typical metal compound and water are mixed, and then, the copper catalyst is added to the resultant mixture.

The present reaction may be carried out under reduced pressure or normal pressure or increased pressure. Preferably, the present reaction is carried out under normal pressure or increased pressure.

A temperature for the present reaction may vary depending on the kind and amount of the copper catalyst to be used etc., and is preferably from 0 to 200° C., more preferably from 50 to 180° C. A reaction temperature not lower than 0° C. tends to permit a higher rate of the oxidation reaction. A reaction temperature not higher than 200° C. tends to higher selectivity for the oxidation reaction.

The reaction time may vary depending on the reaction temperature, the reactants to be used or the like, and is usually from 0.5 to 50 hours.

The degree of the present reaction progress can be confirmed by analytic means such as gas chromatography, high-performance liquid chromatography, thin-layer chromatography, nuclear magnetic resonance spectroscopy, infrared absorption spectroscopy or the like.

After completion of the reaction, the sulfur-containing amino acid may be brought out by a procedure in which the resultant reaction mixture is filtered to remove the copper catalyst therefrom, and then, the reaction mixture is optionally washed with a solvent immiscible to water, and then, neutralized with mineral acid such as sulfuric acid, hydrochloric acid, carbonic acid or the like and is then concentrated and cooled. If the sulfur-containing amino acid is a lipophilic compound, the sulfur-containing amino acid may be brought out by a procedure in which the resultant reaction mixture is filtered to remove the copper catalyst and is then mixed with a solvent immiscible to water, and the resultant mixture is extracted, neutralized, concentrated and cooled. The solvent immiscible to water includes ester solvents such as ethyl acetate, and ether solvents such as methyl tert-butyl ether. The amount of the immiscible solvent to be used is not limited.

The sulfur-containing amino acid thus brought out may be purified by distillation, column chromatography, crystallization or the like.

The sulfur-containing amino acid thus obtained is α-amino acid having a sulfur-containing hydrocarbon group at position 2.

The sulfur-containing amino acid is preferably represented as follow:

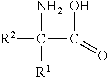

(In the formula, R¹ and R² are defined as above.)

Examples of such an amino acid include 2-amino-3-(methylthio)propionic acid, 2-amino-3-(tert-butylthio)propionic acid, 2-amino-3-(benzylthio)propionic acid, 2-amino-3-(ethylthio)propionic acid, 2-amino-4-(methylthio)butyric acid (i.e., methionine), 2-amino-4-(ethylthio)butyric acid, 2-amino-4-(propylthio)butyric acid, 2-amino-4-(benzylthio)butyric acid, 2-amino-5-(methylthio)pentanoic acid, 2-amino-3-(ethylthio)pentanoic acid, 2-amino-3-(propylthio)pentanoic acid and 2-amino-3-(benzylthio)pentanoic acid.

### EXAMPLES

Hereinafter, the present invention will be described in more detail by way of Examples.

### Example 1

A 50 mL pressure reaction tube equipped with a magnetic rotor was charged with 2-amino-4-methylthio-1-butanol (200 mg), sodium hydroxide (90 mg) and water (2 g), the mixture was stirred. A sponge copper (Raney (trade mark) type, product of Strem Chemical Inc.)(40 mg) was added to the mixture as a developing catalyst. After replacement of the interior of the reaction tube with nitrogen, the resulting mixture was stirred at 140° C. for 8 hours. The reaction mixture was cooled to room temperature, and then, the cooled reaction mixture was filtered to remove the sponge copper therefrom. The resulting filtrate was neutralized with 0.1N sulfuric acid, and water was distilled off to obtain 2-amino-4-(methylthio)butyric acid.

### Determination of Yield

Methanol (5 g) was added to the obtained 2-amino-4-(methylthio)butyric acid, and a 10% hexane solution of trimethylsilyldiazomethane was further added thereto, to obtain methyl 2-amino-4-(methylthio)butyrate. A methanol solution containing the obtained methyl 2-amino-4-(methylthio)butyrate was analyzed by a gas chromatography internal standard method to determine the yield of methyl 2-amino-4-(methylthio)butyrate from 2-amino-4-methylthio-1-butanol. As a result, the yield was 37%. In other words, 2-amino-4-(methylthio)butyric acid was obtained at a yield of 37% or more from 2-amino-4-methylthio-1-butanol. 49% of 2-amino-4-methylthio-1-butanol used as the starting material was recovered.

### Example 2

A 50 mL pressure reaction tube equipped with a magnetic rotor was charged with 2-amino-4-methylthio-1-butanol (200 mg), sodium hydroxide (120 mg) and water (2 g), the mixture was stirred. A sponge copper (Raney (trade mark) type, product of Strem Chemical Inc.)(50 mg) was added to the mixture as a developing catalyst. After replacement of the interior of the reaction tube with nitrogen, the resulting mixture was stirred at 140° C. for 8 hours. The reaction mixture was cooled to room temperature, and then, the cooled reaction mixture was filtered to remove the sponge copper. Ethyl acetate (5 g) was added to the resulting filtrate to separate oil and water, and thus the lipophilic substances were removed therefrom. Carbonic acid was formed by adding dry ice (CO₂) (5 g) to the water phase, and a solid was precipitated upon stirring. The precipitated solid was filtered and dried to obtain a white powder (130 mg). Then, the obtained powder was analyzed by a liquid chromatography (modified area percentage method). As a result, the content of 2-amino-4-(methylthio) butyric acid was 64% (yield: 38%).

### Example 3

A 50 mL pressure reaction tube equipped with a magnetic rotor was charged with 2-amino-3-benzylthio-1-propanol (200 mg), sodium hydroxide (80 mg) and water (2 g), the mixture was stirred. A sponge copper (Raney (trade mark) type, product of Strem Chemical Inc.)(40 mg) was added to the mixture as a developing catalyst. After replacement of the interior of the reaction tube with nitrogen, the resulting mixture was stirred at 140° C. for 8 hours. The reaction mixture was cooled to room temperature, and then, the cooled reaction mixture was filtered to remove the sponge copper therefrom. The resulting filtrate was neutralized with 0.1N sulfuric acid, and water was distilled off to obtain 2-amino-3-(benzylthio)propionic acid.

### Determination of Yield

Methanol (5 g) was added to the obtained 2-amino-3-(benzylthio)propionic acid, and a 10% hexane solution of trimethylsilyldiazomethane was further added thereto, to obtain methyl 2-amino-3-(benzylthio)propionate. A methanol solution containing the obtained methyl 2-amino-3-(benzylthio)propionate was analyzed by a gas chromatography internal standard method to determine the yield of methyl 2-amino-3-(benzylthio)propionate from 2-amino-3-benzylthio-1-propanol. As a result, the yield was 45%. In other words, 2-amino-3-(benzylthio)propionic acid was obtained at a yield of 45% or more from 2-amino-3-benzylthio-1-propanol.

45% of 2-amino-3-benzylthio-1-propanol used as the starting material was recovered.

### INDUSTRIAL APPLICABILITY

The present invention is industrially applicable as a process for producing the sulfur-containing amino acids such as methionine.

1. A process for producing a sulfur-containing amino acid, comprising a step of oxidizing a 2-aminoethanol compound having, at position 2, a sulfur-containing hydrocarbon group having 1 to 24 carbon atoms in the presence of copper and water.

2. The process according to claim 1, wherein the step of oxidizing the 2-aminoethanol compound is carried out further in the presence of at least one typical metal compound selected from the group consisting of alkali metal compounds and alkaline earth metal compounds.

3. The process according to claim 2, wherein the typical metal compound is at least one compound selected from the group consisting of alkali metal hydroxides and alkali earth metal hydroxides.

4. The process according to claim 1, wherein the sulfur-containing hydrocarbon group has no non-aromatic multiple bond.

5. The process according to claim 1, wherein the 2-aminoethanol compound is 2-amino-4-methylthio-1-butanol.
